(19) United States
(12) Patent Application Publication
(10) Pub. No.: US 2026/0325174 A1
(43) Pub. Date: Oct. 22, 2026

(54) SEMINIFEROUS TUBULES EXTRACTION DEVICE

(71) Applicant: UNIVERSITÀ DEGLI STUDI ENNA "KORE", Enna (IT)

(72) Inventor: Rosario LEONARDI, Enna (IT)

(73) Assignee: UNIVERSITÀ DEGLI STUDI ENNA "KORE", Enna (IT)

(21) Appl. No.: 19/486,415

(22) PCT Filed: May 31, 2024
(86) PCT No.: PCT/IB2024/055360

§ 371 Date: Nov. 21, 2025

(30) Foreign Application Priority Data
May 31, 2023 (IT) 102023000011046

(51) Int. Cl. A61B 17/43; A61B 17/00; A61B 17/32; A61B 17/3205; A61B 17/34

(52) U.S. Cl. CPC A61B 17/43; A61B 17/32053; A61B 2017/00424; A61B 2017/320064; A61B 2017/3454; A61B 2217/005

(57) ABSTRACT

A device for extracting seminiferous tubules comprises a hollow cylindrical body having at one end a seat connectable to a suction device; and a tip at the opposite end of the seat; wherein the tip has an ogival shape inside which is included a concave zone with a radius of curvature contained in a plane to which belongs the axis of the cylindrical body, thereby defining a spoon or cup shape capable of accommodating a seminiferous tubule and allowing, when the extraction device is under negative pressure via a suction device connected to the seat, to retain at least the seminiferous tubule inside the concave zone.

### CROSS REFERENCE TO RELATED APPLICATIONS

This application is a national stage entry of International Application No. PCT/IB2024/055360, filed on May 31, 2024, which is based upon and claims foreign priority to Italy Patent Application No. 102023000011046, filed on May 31, 2023, the entire contents of which are incorporated herein by reference.

### TECHNICAL FIELD

The present invention relates to the field of medical instruments. More specifically, the present invention relates to a device for the extraction of seminiferous tubules via percutaneous approach and the related extraction method.

### BACKGROUND

It is known from studies and research conducted by the World Health Organization that an increasing number of couples of reproductive age are affected by fertility problems. Azoospermia is the most severe form of male infertility. Patients who still maintain spermatozoa production, even if not released externally (due to low numbers or obstruction of the seminal ducts), can undergo surgical extraction to obtain germ cells from the testicle or epididymis (TESE and Micro TESE). The procedure, which involves an incision of the tissues covering the testicle, must be sufficiently wide to allow exploration of the organ, which is incised to extract the seminiferous tubules. The TESE and Micro-TESE procedures are, therefore, invasive procedures that require the incision of the cutaneous and subcutaneous layers covering the testicle and the patient's testicular tunica albuginea to extract the seminiferous tubules. Often, before proceeding with open surgical intervention, an attempt is made to aspirate the sperm through the puncture of the testicle via the skin (TESA). The TESA procedure is less invasive than the previous ones since it is performed by percutaneous aspiration, where the needle, once penetrated the skin, is placed in aspiration through a syringe connected to it. With this technique, an attempt is made to aspirate the sperm contained within the organ. However, the TESA procedure, especially in non-obstructive azoospermia, allows for the collection of only modest quantities of spermatozoa, forcing the patient to undergo repeated punctures, often leading to the actual biopsy.

### SUMMARY

The purpose of the present invention is to resolve, at least in part, the aforementioned disadvantages by providing a medical device that allows for the extraction of seminiferous tubules from the testicles without the need for surgical exploration of the scrotum. The purpose of the present invention is achieved by a device for the extraction of seminiferous tubules comprising:
a hollow cylindrical body having a seat at one end that can be connected to an suction device;
a tip positioned at the opposite end of the seat; characterized by the fact that this tip has an ogival shape, within which a concave area is included, having a radius of curvature contained in a plane to which belongs the axis of the cylindrical body, and which allows, when the extraction device is placed under negative pressure, by means of an suction device connected to the seat, to retain at least one seminiferous tubule within said concave area. Thanks to its particular shape, combined with a simplified procedure that does not require the incision of tissues, the device of the present invention minimizes the invasiveness of the operation for extracting seminiferous tubules from one or both testicles, even in different parts of the organ if the extracted tubules do not contain spermatozoa. Additionally, the extraction procedure is economical compared to other extraction and/or aspiration procedures since all the elements used are simple to construct, single-use, and of low cost. According to a preferred embodiment, the tip includes a hooked terminal part designed to anchor the tubules by rotating the extraction device when inside the testicular tissue and to keep the seminiferous tubule in traction once hooked during its extraction from the testicle. In a further preferred embodiment, the edges of the ogival-shaped tip are sharp to allow the cutting of the tubule anchored inside a testicular lobule. Preferably, the hollow cylindrical body is curved at an angle between 15 and 20 degrees to facilitate the positioning of the device relative to the surface of the testicle. Preferably, the extraction device includes a pair of fins near the seat. Preferably, the diameter of the hollow cylindrical body is 19 Gauge (0.912 mm). In another embodiment, the extraction device can be part of a low-cost extraction kit. Other advantages of the present invention are discussed in the description and cited in the dependent claims.

### BRIEF DESCRIPTION OF THE DRAWINGS

The invention is described below based on non-limiting examples illustrated for illustrative purposes in the following figures, which respectively refer to:

FIG. 1 shows the device of the present invention in a top view.

FIG. 2 shows the device of the present invention in a side view.

FIG. 2b shows a view of the device from FIG. 2 highlighting the hooked terminal part.

FIG. 3 shows the device of the present invention during a phase of extracting a tubule from a testicle.

FIG. 4 shows the anatomy of a testicle.

### DETAILED DESCRIPTION OF THE EMBODIMENTS

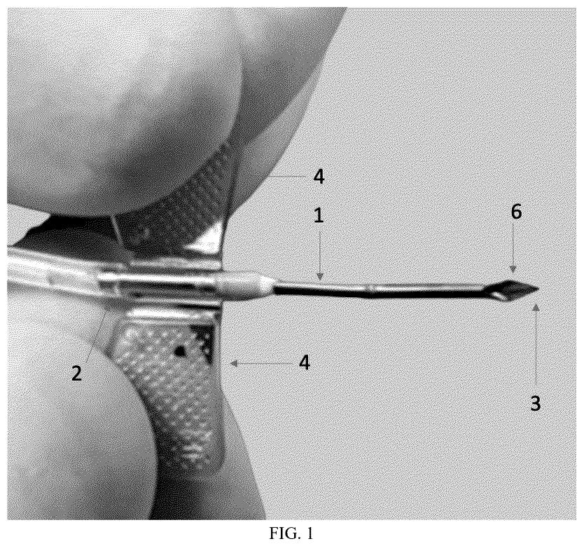

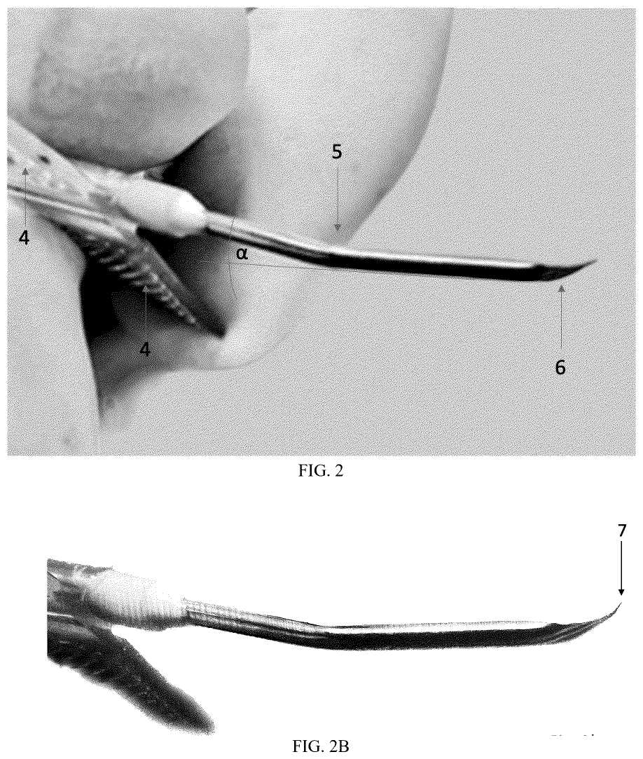

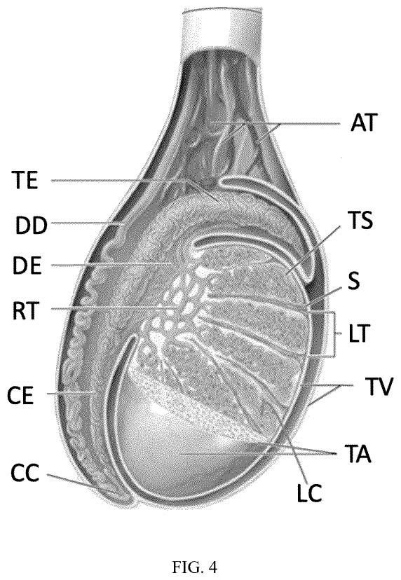

In FIG. 1, the extraction device according to one embodiment of the present invention is shown. The device for extracting the seminiferous tubules, shown in FIG. 4 with the reference TS, comprises a hollow cylindrical body 1 having a seat 2 at one end that can be connected to an aspiration system through a connecting tube made of a compressible material, preferably PVC. The extraction device also includes a tip 3 characterized by having an ogival shape, within which a concave area 6 is included. As will be better explained below, this concave area 6 allows for the reception and retention of at least one seminiferous tubule TS inside it. The extraction device near the seat 2 further comprises a pair of fins 4 to increase the grip surface by an operator and facilitate the rotation of the device once it has reached a testicular lobule, shown in FIG. 4 with the reference LT. These fins are preferably made of PVC and include a plurality of knurling to increase friction, making them easily manipulated by the hands of an operator. In FIG. 2, the extraction device is shown in a side view, making other characteristics of the device more evident. In particular, it can be observed that the cylindrical body 1 has a specific curvature angle a 5 that aids the operator during the positioning of the device relative to the curved surface of the testicle. Preferably, the curvature angle a 5 has a value between 15 and 20 degrees. Preferably, the diameter of the hollow cylindrical body (1) is 19 Gauge, that is 0.912 mm.

In FIG. 2, the concave area 6, which has the hooked tip 3 of the extraction device, is highlighted, particularly how this concave area has a radius of curvature contained in a plane to which the axis of the cylindrical body 1 belongs. This concave area 6 thus defined forms a sort of ‘spoon or cup’ capable of hosting at least one tubule TS, preventing it from slipping away during extraction. Another feature observable in FIG. 2B is the terminal part of the tip 3, which ends with a hooked shape 7. This particular shape of the terminal part allows anchoring the tubules TS and maintaining them under tension during the extraction from the testicle. Specifically, the hooked terminal part 7 allows anchoring the seminiferous tubules TS through a rotational movement of the extraction device when the tip 3 is inside the testicular tissue, keeping it under tension during the extraction phase from the testicle. To facilitate this operation, the concave area 6 has a cross-section with a first maximum dimension, and the cylindrical body 1 has a cross-section with a second maximum dimension, preferably a maximum diameter, where the first maximum dimension is greater than the second maximum dimension, to increase the surface area on which at least one seminiferous tubule is deposited during the extraction operation and to favour the cutting of the tubule during rotation. The following paragraph briefly describes the anatomy of a testicle shown in FIG. 4. The testicle has an ovoid shape and is surrounded by two tunics: the tunica vaginalis TV and the tunica albuginea TA. From the tunica albuginea TA, a septum S, called the mediastinum, departs, dividing the testicles into 250-300 wedge-shaped compartments called testicular lobules LT. Each lobule LT contains 1 to 4 seminiferous tubules TS. The seminiferous tubules TS converge to form a straight tubule for each lobule LT, leading the spermatozoa to the rete testis RT, that is a network of tubules from which the efferent ducts DE depart. These efferent ducts DE exit the testicle and lead the spermatozoa to the epididymis, located on the outer surface of the testicle and divided into three zones: the head of the epididymis TE, which receives the efferent ducts DE, the body of the epididymis CE, and the tail of the epididymis CC, which ends in the vas deferens DD. The blood supply to the testicles is provided by the testicular artery AT. Finally, the curved line LC visually represents one of the possible sites where the device of the present invention can penetrate the testicle for the extraction of seminiferous tubules. As previously mentioned, the procedure for extracting seminiferous tubules TS with the extraction device is a simplified procedure that minimizes invasiveness to the patient. To perform the procedure for extracting seminiferous tubules TS, in addition to the extraction device subject of the present invention, an suction device, preferably a 10 ml syringe, a flexible connecting tube to connect the syringe to the seat 2 of the extraction device, and a clamp are also used. The procedure for extracting seminiferous tubules TS involves the extraction device, thanks to its particular curvature 5, easily reaching the area of the testicle identified as the extraction site. The hooked terminal part 7 of the device is introduced perpendicularly to the skin of the scrotum and advanced until it passes the tunica albuginea TA of the testicle. Once the testicular lobule LT is reached, the device is rotated 15-20 degrees so that the part protruding from the skin of the scrotum assumes a direction perpendicular to the insertion site due to its curved shape. At this point, the device is rotated while the syringe is activated to generate negative aspiration pressure in the area near the tip 3. This condition of negative aspiration pressure will henceforth be referred to as the system under aspiration. To maintain this system under aspiration for the rest of the extraction procedure without the operator continuing to act on the syringe, the connecting tube is closed with a clamp. The rotational movement of the extraction device allows the tip 3 inside the testicular lobule LT to cut the tubules TS using the sharp edges of the ogival-shaped tip 3. Once cut, the seminiferous tubules are aspirated into the concave area 6 of the tip 3 due to the negative pressure, remaining anchored to the hooked terminal part 7. At this point, the extraction phase continues, keeping the tip 3 in contact with the internal tunica albuginea TA of the testicle so that the tubule TS is retained, preventing it from slipping away from the extraction device. Continuing with the extraction action, when the tip 3 exits the testicle, the tubule TS is hosted and retained within the concave area 6 due to the aspiration, and, as mentioned, the hooked terminal part 7 allows maintaining the tubule under tension once outside the testicle.

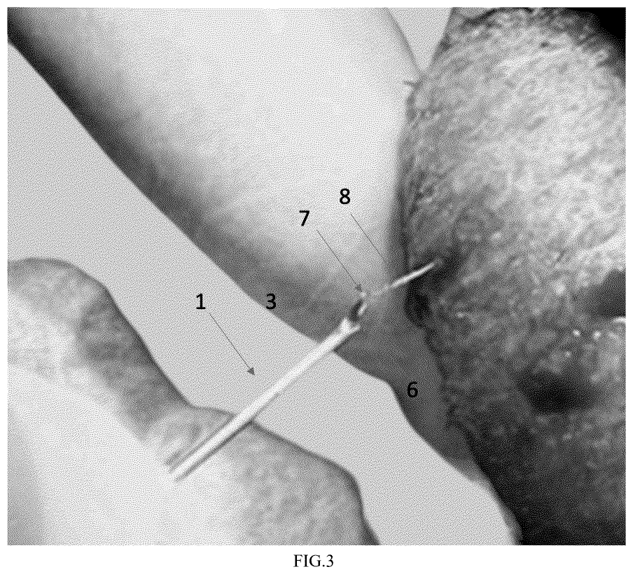

In FIG. 3, the final phase of extracting the seminiferous tubule TS is shown, wherein it is possible to see the tubule TS protruding 8 from the testicle, still anchored and under tension at the hooked terminal part 7 of the tip 3. At this point, using forceps, the tubule TS is collected and placed on a plate, after which a biologist will analyse it and extract the spermatozoa contained within it. This extremely simple and quick procedure allows for multiple tubule extractions until the biologist deems the collected spermatozoa sufficient for ICSI, which is the laboratory technique that allows the insertion of a single spermatozoon into a mature oocyte. The repeatability of the extraction phase within the same session is also possible because this procedure does not require any incisions of the testicles. As a result, the patient's healing times are also shorter. As described previously, this procedure is minimally invasive thanks to the particular shape of the extraction device, additionally, using other simple medical instruments, it is possible to provide an extraction kit that can be produced at low cost precisely due to the selection of these medical instruments. This kit, in addition to comprising the extraction device, may also include one or more elements such as: an suction device, preferably a 10 ml syringe, a connecting tube, preferably made of PVC, to connect the suction device to the seat 2 of the extraction device, and a clamp made preferably of PVC.

1. A device for extracting seminiferous tubules comprising:
a hollow cylindrical body having at one end a seat connectable to a suction device;
a tip at the opposite end of the seat; wherein the tip has an ogival shape inside which is included a concave zone with a radius of curvature contained in a plane to which belongs the axis of the cylindrical body, defining a spoon or cup shape capable of accommodating a seminiferous tubule and allowing, when the extraction device is under negative pressure via a suction device connected to the seat, to retain at least the seminiferous tubule within the concave zone.

2. The device for extracting seminiferous tubules according to claim 1, wherein the tip has a hooked terminal part such that at least one seminiferous tubule remains anchored and under tension during extraction from the testicle.

3. The device for extracting seminiferous tubules according to claim 1, wherein the edges of the ogival-shaped tip are sharp to allow for the excision of at least one tubule anchored inside the testicular lobule.

4. The device for extracting seminiferous tubules according to claim 1, wherein the hollow cylindrical body is curved at an angle between 15 and 20 degrees to facilitate the positioning of the device relative to the surface of the testicle.

5. The device for extracting seminiferous tubules according to claim 1, wherein the hollow cylindrical body has a diameter of 19 Gauge.

6. The device for extracting seminiferous tubules according to any claim 1, wherein the device comprises a pair of fins near the seat to increase the gripping surface of the device by an operator.

7. The device for extracting seminiferous tubules according to claim 1, wherein the concave zone has a cross-section,
a first maximum dimension, and the cylindrical body has a cross-section with a second maximum dimension, wherein the first maximum dimension is greater than the second maximum dimension.

8. A kit for extracting seminiferous tubules comprising an device for extracting seminiferous tubules according to claim 1, wherein the kit further comprises a suction device and a connecting tube, preferably made of PVC, for connecting the suction device to the seat of the device for extracting seminiferous tubules so that the device for extracting seminiferous tubules is placed under suction.

9. The kit according to claim 8, wherein the suction device is a 10 ml syringe.

10. The kit according to claim 8, wherein the kit further comprises a clamp, preferably made of PVC, to close the connecting tube to maintain the extraction device under suction.

11. The device for extracting seminiferous tubules according to claim 2, wherein the edges of the ogival-shaped tip are sharp to allow for the excision of at least one tubule anchored inside the testicular lobule.

12. The device for extracting seminiferous tubules according to claim 2, wherein the hollow cylindrical body is curved at an angle between 15 and 20 degrees to facilitate the positioning of the device relative to the surface of the testicle.

13. The device for extracting seminiferous tubules according to claim 3, wherein the hollow cylindrical body is curved at an angle between 15 and 20 degrees to facilitate the positioning of the device relative to the surface of the testicle.

14. The device for extracting seminiferous tubules according to claim 2, wherein the hollow cylindrical body has a diameter of 19 Gauge.

15. The device for extracting seminiferous tubules according to claim 3, wherein the hollow cylindrical body has a diameter of 19 Gauge.

16. The device for extracting seminiferous tubules according to claim 4, wherein the hollow cylindrical body has a diameter of 19 Gauge.

17. The device for extracting seminiferous tubules according to claim 2, wherein the device comprises a pair of fins near the seat to increase the gripping surface of the device by an operator.

18. The device for extracting seminiferous tubules according to claim 3, wherein the device comprises a pair of fins near the seat to increase the gripping surface of the device by an operator.

19. The device for extracting seminiferous tubules according to claim 4, wherein the device comprises a pair of fins near the seat to increase the gripping surface of the device by an operator.

20. The device for extracting seminiferous tubules according to claim 5, wherein the device comprises a pair of fins near the seat to increase the gripping surface of the device by an operator.
